(19) United States
(12) Patent Application Publication
(10) Pub. No.: US 2020/0323707 A1
(43) Pub. Date: Oct. 15, 2020

(54) ABSORBENT PRODUCT HAVING NANO-COATING LAYER, AND MANUFACTURING METHOD THEREFOR

(71) Applicants: Jae-ho RHO, Seoul (KR); Young-ho RHO, Seoul (KR); Joo-ri RHO, Seoul (KR); Jung-ho HWANG, Seoul (KR)

(72) Inventors: Jae-ho RHO, Seoul (KR); Young-ho RHO, Seoul (KR); Joo-ri RHO, Seoul (KR); Jung-ho HWANG, Seoul (KR)

(21) Appl. No.: 16/097,595

(22) PCT Filed: Apr. 29, 2016
(86) PCT No.: PCT/KR2016/004562

§ 371 Date: Oct. 29, 2018

(51) Int. Cl. A61F 13/511; A61F 13/472; A61F 13/49; A61F 13/15

(52) U.S. Cl. CPC A61F 13/51121; A61F 13/472; A61F 2013/8402; A61F 13/15577; A61F 13/49007

(57) ABSTRACT

The present invention relates to a disposable absorbent product such as a disposable diaper, a sanitary napkin, and the like and, more specifically, to: an absorbent product having a nano-coating layer, which is formed on the outer surface of a disposable absorbent product for absorbing excrement to be excreted from the body, thereby enabling overall air permeability of the product to improve and moisture to be blocked; and a manufacturing method therefor. To this end, in the absorbent product making contact with the body so as to absorb and store the excrement and the like to be excreted or discharged from the body, the present invention is characterized in that a nano-coating layer is formed on the outer surface, exposed to the outside, of the absorbent product in order to prevent the absorbed and stored moisture from being discharged to the outside and to ensure air permeability allowing outside air to circulate inside.

### TECHNICAL FIELD

The present invention relates to a disposable absorbent product such as a disposable diaper, a sanitary napkin etc. and more specifically, to an absorbent product having a nano-coating layer, which is formed on the outer surface of a disposable absorbent product for absorbing body waste to be excreted from the body, thereby making it possible to improve overall air permeability of the product and to block moisture, and a manufacturing method therefor.

### DESCRIPTION OF THE RELATED ART

In general, disposable absorbent products such as a baby diaper or an adult diaper, a sanitary napkin, a panty liner etc. are used to handle body waste excreted through physiological activity from the body. These disposable absorbent products absorb body waste containing moisture in the state of directly contacting the body. If a wet product is kept for too long a period, part of the skin that contacts the wet product having absorbed moisture containing ammonia gets irritated and becomes prone to infections in worse cases. Thus, absorbing power needs to be ensured so as to absorb moisture and then store the same, and air permeability needs to be ensured so as to allow passage of external air.

A diaper is a typical example of disposable absorbent products. South Korean Utility Model Registration No. 20-257960 (registered on Dec. 24, 2001), as a related art, discloses a diaper. Traditional diapers including a diaper according to the related art are arranged to contact the skin of the body and includes a pad whose front and rear ends protrude in front and rear directions of the waist of a user, and fixing means provided at the front and rear ends of the pad so as to fix the pad to be worn around the waist of the user.

Additionally, the pad includes an upper sheet that contacts the body of a user and allows body waste such as liquid waste and solid waste to permeate, an absorbing pad provided inside the pad, consisting of absorbent materials and configured to absorb and store body waste such as liquid waste or solid waste etc., and a waterproof sheet provided under the absorbing pad so as to prevent clothes from being dirtied. In this case, the upper sheet consists of any one of a synthetic resin, synthetic fiber, natural fiber so as to allow excrement such as liquid waste to permeate.

Further, the fixing means consist of Velcro-type elastic belts provided at the front and rear ends of the pad.

An advertising copy, a figure, a trademark etc. are printed on an external surface of the waterproof sheet, which is exposed to the outside, the absorbing pads are piled on an inner surface of the waterproof sheet, and the waterproof sheet consists of pulp, synthetic fiber etc.

Traditional diapers with the above-described configuration absorbs and stores moisture contained in body waste through the absorbing pad and blocks the moisture from being discharged to the outside with the waterproof sheet. However, a traditional waterproof sheet is directed to preventing moisture from escaping. Thus, it is hard for them to allow external air to come into a diaper and help improve air permeability.

As a means to solve such problems, a so-called “breathing diaper” is on the market. As an example, there is a diaper that has a corrugation part at limited portions thereof or a diaper that consists of microfibers.

However, air permeability may be ensured only at some portions of such a traditional product. Thus, the body that contacts an absorbing pad gets irritated in the state where the absorbing pad stores a large amount of moisture.

Further, disclosed is a product that prevents germs from growing in moisture and body waste stored in an absorbing pad by means of the application of a silver solution or silver powder to the absorbing pad. However, the silver solution or power does not adhere to the upper sheet or the absorbing pad strongly and therefore a layer of the silver solution or silver powder is easily taken off. Additionally, a silver solution or silver powder is not evenly applied and therefore it is hard to achieve a strong sterilizing effect.

As a means to solve these problems, a produce whose pad is woven from nanofibers has been developed. However, the product is disadvantageous due to an increase in manufacturing costs and in time required for the manufacture of the product.

### DETAILED DESCRIPTION OF THE INVENTION

### Technical Problems

As a means to solve the above-described problems, the present invention relates to an absorbent product having a nano-coating layer, which is formed on the outer surface of a disposable absorbent product, thereby making it possible to improve overall air permeability and to block moisture with the nano-coating layer, and a manufacturing method therefor.

### Technical Solutions

As a means to achieve the above-describe purposes, the present invention is characterized in that an absorbent product, which contacts the body, and absorbs and stores body waste etc. to be excreted or discharged from the body, has a nano-coating layer formed on an external surface of the absorbent product, which is exposed to the outside, so as block absorbed and stored moisture from being discharged to the outside, and to allow passage of external air thereby ensuring air permeability.

In this case, the nano-coating layer is formed by applying 5 nm to 500 nm, preferably 25 nm to 250 nm of a monomer for nano-coating, which is not harmful to the human body.

Further, the nano-coating layer is formed through any one of knife coating, roller coating, gravure coating, and powder coating, extrusion coating, dip coating, rod coating, spray coating, low-pressure plasma processing.

In this case, the low-pressure plasma processing takes place in a vacuum plasma chamber, pressure for performing the processing typically ranges from 10 mTorr to 1000 mTorr, electric power that will be used typically ranges from 5 W to 5000 W, and nano-coating is repetitively performed until the nano-coating layer has a thickness selected from any one of a thickness ranging from 5 nm to 500 nm and preferably, a thickness ranging from 25 nm to 250 nm.

A method for manufacturing an absorbent product having a nano-coating layer according to the present invention includes a sheet transferring step of winding a material selected from materials, which do not cause rashes on the body such as pulp, non-woven fabric, usual fiber etc., around a winding roller 110 in the form of a sheet, and transferring the sheet at constant speed, a primary drying step of heating and drying the transferred sheet 100, a coating step of applying 5 nm to 500 nm, preferably 25 nm to 250 nm of a monomer for nano-coating, which is not harmful to the human body, to one surface of the dried sheet 100, a secondary drying step of drying the sheet whose one surface is nano-coated, and a cutting step of cutting the sheet such that the sheet has a predetermined length or form.

### Advantageous Effects

e present invention configured as described above has a nano-coating layer formed on an outer surface of a disposable absorbent product for absorbing body waste to be excreted from the body, so as to improve overall air permeability of the product and to block moisture, thereby making it possible to prevent the body of a user from getting irritated and infected. Additionally, the cost of manufacturing the present invention is lower than that of a product using microfiber fabric. Thus, the present invention is highly competitive in the market.

### BRIEF DESCRIPTION OF THE DRAWINGS

FIG. 1 is a perspective view of an absorbent product having a nano-coating layer according to the present invention.

FIG. 2 is a sectional view of an absorbent product having a nano-coating layer according to the present invention.

FIG. 3 is a perspective view of another embodiment of an absorbent product having a nano-coating layer according to the present invention.

FIG. 4 is a flow chart illustrating a process of manufacturing a nano-coating layer according to the present invention.

FIG. 5 is a schematic view illustrating the process according to the flow chart in FIG. 4.

FIG. 6 is a flow chart illustrating another process of manufacturing a nano-coating layer according to the present invention.

FIG. 7 is a schematic view illustrating the process according to the flow chart in FIG. 6.

### MODE FOR CARRYING OUT THE INVENTION

Preferred embodiments of an absorbent product having a nano-coating layer according to the present invention and a manufacturing method therefor will be described in detail with reference to the attached drawings.

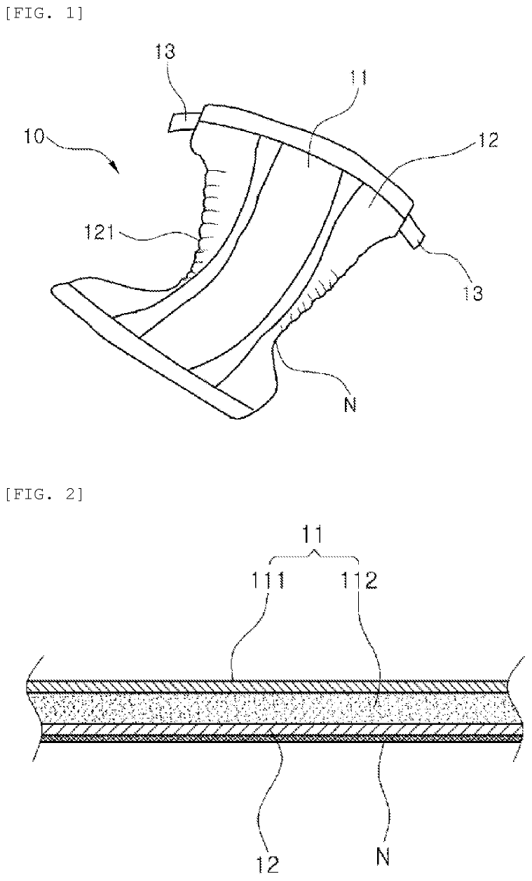

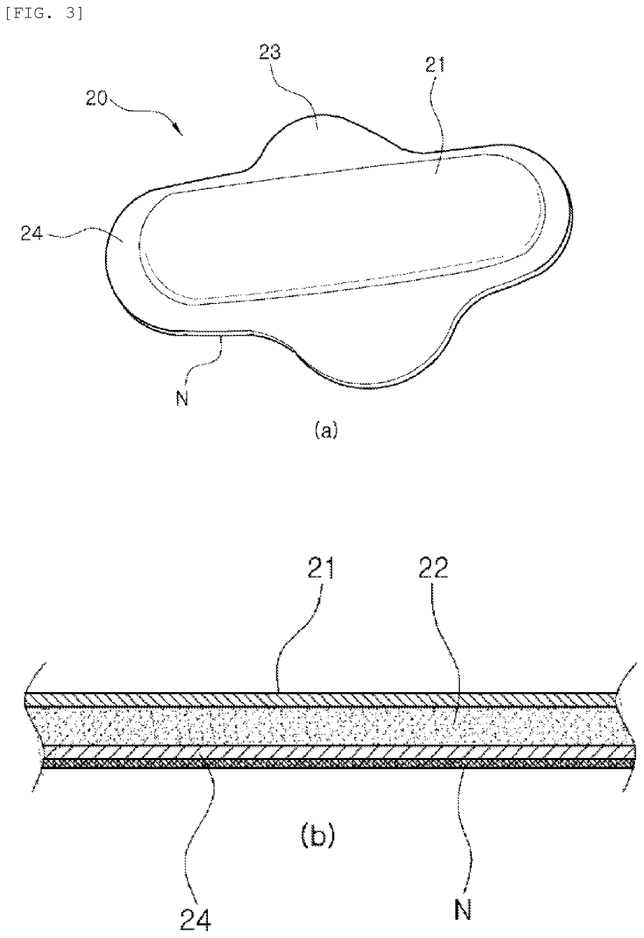

FIGS. 1 to 2 illustrate an embodiment of a disposable diaper having a nano-coating layer according to the present invention among disposable absorbent products. The disposable diaper 10 includes a pad 11 which contacts the body, an outer surface 12 having an inside to which the pad 11 is attached and having front and rear ends which are longer than those of the pad 11 so as to be arranged at the front and rear of the waist of a user, and fixing means 13 detachably arranged at the front and rear ends of the outer surface 12. The outer surface 12 has a nano-coating layer N.

The pad 11 includes a liquid-permeable upper sheet 111, and an absorbent pad 112 for absorbing and storing liquid, which is arranged under the upper sheet 111 and connected at least partially to the upper sheet 111, and the upper sheet 111 preferably has an arc shape so as to come into close contact with a user.

The outer surface 12 has an inside to which the pad 11 is integrally attached, a surface area which is larger than that of the pad 11, and a front end and a rear end which are longer than those of the pad 11 so as to be arranged at the front and the rear of the waist of a user. In this case, the pad 11 arranged at an inner surface of the outer surface 12 has a corrugation part 121 of a predetermined length on both lateral portions thereof.

The fixing means 13 are provided at the front and rear ends of the outer surface 12 so as to fix the outer surface 12 to the waist of a user. The fixing means has a Velcro-type attaching surface on one surface thereof such that the attaching surface is detachably fixed to an external surface of the outer surface.

The above-described disposable diaper 10 has a usual configuration, and thus the structure will not be described in detail.

A nano-coating layer N according to the present invention is arranged on the outer surface 12 of a disposable diaper 10. Five nm to five hundred nm, preferably 25 nm to 250 nm of a monomer for nano-coating, which is not harmful to the human body, is applied to the surface area of a material selected from materials that do not cause rashes to the skin such as pulp, nonwoven fabric, usual fabric etc., and then is formed and attached to the outer surface 12 of a disposable diaper 10.

To form the nano-coating layer N, one surface of any one of pulp, nonwoven fabric, usual fiber is processed. The processing method includes knife coating, roller coating, gravure coating, powder coating, extrusion coating, dip coating, rod coating, spray coating etc. Each of the processing methods may be selectively applied depending on a material of the outer surface 12 of a disposable diaper 10.

The above-described monomer for nano-coating refers to a basic material constituting high-molecular compounds, polymers. According to the present invention, a monomer used as a starting material is preferably introduced into plasma in the form of a gas. The monomer is easily introduced into a plasma chamber used in the low-pressure plasma processing at a nano-coating step that will be described below, a liquid precursor or a solid precursor may be selectively used as a monomer at atmospheric pressure or at reduced pressure, and the monomer is typically evaporated within the temperature of 200° C. with simple heating.

The basic material preferably contains halogen and/or phosphorus and/or nitrogen and/or silicon. For instance, there are monomers obtained from one or more precursor CF₄, C₂F₆, C₃F₆, C₃F₈, C₄F₈, C₃F₆, C₅F₁₂, C₆F₁₄ and/or saturated or unsaturated hydro fluorocarbons (CxFy), obtained from acrylates (e.g. C₁₃H₁₇O₇F₂), methacrylates (e.g. C₁₄H₉F₁₇O₂) or a mixture thereof, obtained from trimethyl phosphate, triethyl phosphate, tripropyl phosphate or one or more precursors of derivatives of other phosphoric acids, obtained from one or more precursors of ethylamine, triethylamine, allylamine or acrylonitrile, or, obtained from siloxane, silane or a mixture thereof etc.

In addition to a method in which a nano-coating layer N is directly formed on an outer surface 12, a nano-coating layer N may be separately formed and then attached to an outer surface 12. In this method, a nano-coating layer N is formed on one surface of any one selected from pulp, nonwoven fabric, usual fiber via roller coating and cut so as to have a size corresponding to that of an outer surface 12 to which the nano-coating layer will be attached. Then the cut nano-coating layer N is attached to the outer surface 12 via heat adhesion or ultrasonic welding, dried and integrally attached to the outer surface.

By doing so, air permeability may be ensured through a portion where the nano-coating layer is formed. In this case, a nano-coating layer is formed on the entire surface of an outer surface 12 or at least partially formed on the outer surface. Therefore, unlike a traditional diaper whose corrugation parts are formed on both lateral portions of a pad so as to ensure air permeability, absorbent products according to the present invention may ensure air permeability through a wide surface and various portions.

That is, in terms of traditional diapers, air permeability may be partially ensured, while in terms of the present invention, air permeability may be ensured through the entire surface of a nano-coating layer N. In this case, a nano-coating layer N may be formed at the entire surface of the outer surface 12 of a diaper when necessary. Therefore, absorbent products according to the present invention enable air to circulate through a wider surface area than traditional products.

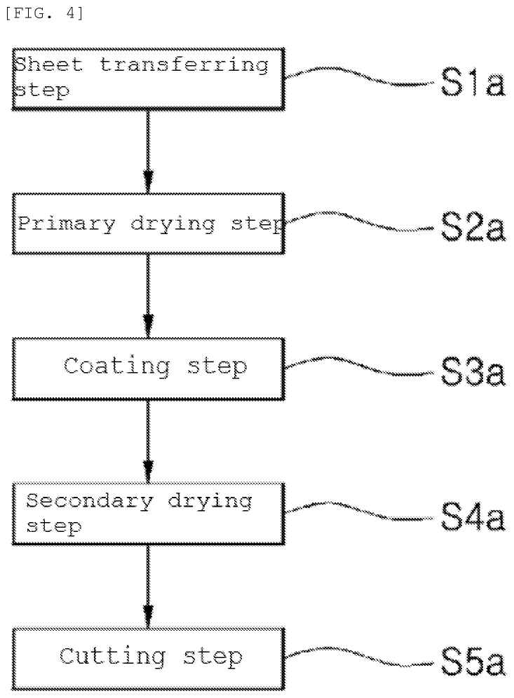

FIG. 3 illustrates another embodiment of an absorbent product having a nano-coating layer N according to the present invention, and more specifically, a female sanitary napkin 20 having a nano-coating layer N formed on an external surface thereof.

A female sanitary napkin 20 includes a liquid-permeable upper sheet 21 which contacts the body, an absorbent pad 22 for absorbing and storing body waste, which is arranged under the upper sheet 21, and an outer sheet 24 having an inner surface to which the absorbent pad 22 is arranged and coupling means 23 detachably coupled to part of an external surface and underwear.

The female sanitary napkin 20 is provided with a nano-coating layer N according to the present invention except for part of the external surface with which the coupling means 23 provided, so as to ensure air permeability. In particular, when a female sits on a chair while wearing a female sanitary napkin 20, water is blocked from being discharged to the outside and air permeability is ensured through a nano-coating layer N.

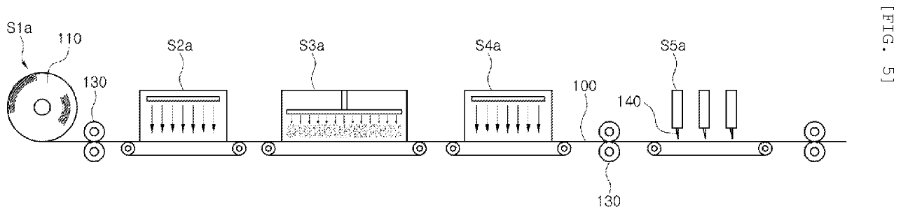

FIG. 4 is a flow chart illustrating an embodiment of a process of forming a nano-coating layer according to the present invention. The process includes a sheet transferring step S1a of winding a material selected from materials, which do not cause rashes on the body such as pulp, non-woven fabric, usual fiber etc., around a winding roller 110 in the form of a sheet, and transferring the sheet at constant speed, a primary drying step S2a of heating and drying the transferred sheet 100, a coating step S3a of applying 5 nm to 500 nm, preferably 25 nm to 250 nm of a monomer for nano-coating, which is not harmful to the human body, to one surface of the dried sheet 100, a secondary drying step S4a of drying the sheet whose one surface is nano-coated, and a cutting step S5a of cutting the sheet such that the sheet has a predetermined length or form.

The sheet transferring step S1a includes winding a sheet 100, where a material that do not cause rashes on the body such as pulp, non-woven fabric, usual fiber etc. is formed into thin cloth, around a winding roller 110, and then transferring the sheet at constant speed through a conveyor belt 120 and a plurality of support rollers 130 controlled by a control part (invisible).

In this case, the speed at which the conveyor belt 120 transfers the sheet is controlled so as to correspond to the speed of the primary drying, secondary drying, coating and cutting steps, and the plurality of support rollers 130 are arranged in pairs in the up-down direction such that a pair of support rollers face each other, thereby making it possible to maintain the tension of the sheet 100.

Further, an advertising copy, a figured etc. are preferably printed in advance on one surface of a sheet 100, more specifically, on a surface exposed to the outside.

Next, the primary drying step S2a includes heating one surface of the sheet 100 and drying the sheet 100 such that a nano-coating material can be strongly attached to the sheet 100. In this step, the sheet 100 is dried with a heating means such as hot air, hot wire etc. In this case, temperature is controlled depending on the material of the sheet 100. The sheet is heated at a temperature ranging from 5° C. to 200° C., preferably ranging from 20° C. to 90° C.

Next, the coating step S3a includes evenly applying a nano-coating material to the dried and transferred sheet 100. The applying method includes knife coating, roller coating, gravure coating, powder coating, extrusion coating, dip coating, rod coating, spray coating etc. In the present invention, a low-pressure plasma processing method among various coating methods will be described as an example.

The low-pressure plasma processing is useful to form a nano-coating layer that is 5 nm to 500 nm thick and to evenly coat every portion, and is appropriate for a small structure with a complex shape and form. Therefore, the low-pressure plasma processing is useful to form a 25-to-250-nm-thick nano-coating layer required for the present invention.

The low-pressure plasma processing takes place in a vacuum plasma chamber. Herein, a parameter for controlling this processing includes electric power, pressure, temperature, the type of a monomer, flow, frequency of a plasma generator, and processing time. The frequency of a plasma generator may range from kHz, to MHz and to GHz, and may be a pulse mode or a continuous mode. The number and arrangement of electrodes are also variable.

Additionally, pressure for performing the low-pressure plasma processing typically ranges from 10 mTorr to 1000 mTorr. The processing is performed until the nano-coating layer has a predetermined thickness. Electric power that will be used heavily depends on a monomer that will be use, but typically, may change between 5 W to 5000 W. Further, pulse-type power or continuous type power may be supplied. In a pulse-type power mode, the pulse repetition interval typically ranges from 1 Hz to 100 kHz, and the mark space ratio typically ranges from 0.05 to 50%.

Further, a surface is covered with a particle that may be polymerized from a plasma-forming gas to form a coating layer. A monomer used as a starting material is introduced into plasma in the form of a gas. This stars with glow discharge. Excited electrodes generated in glow discharge ionize monomer molecules. The monomer molecules decompose while generating free electrons, ions, excited molecules, and radicals. Radicals are absorbed, condensed and polymerized on a sheet. The electrons and ions form a cross-linked bond, or form a chemical bond with a material coated on a surface of the sheet. Preferably, free radicals are generated using a monomer used in plasma polymerization processing.

Next, the secondary drying step S4a includes heating the sheet 100 coated with a nanomaterial such that the nano-coating material is strongly attached to the sheet 100. Temperature is controlled depending on the materials of the sheet and nano-coating. The temperature for heating the sheet ranges from 5° C. to 200° C. and preferably ranges from 20° C. to 90° C.

Next, the cutting step S5a includes cutting the sheet 100 depending on the sorts and forms of absorbent products. The sheet may be cut many times so as to have a desired shape.

That is, a plurality of cutter razors 140 are consecutively arranged with respect to the direction in which the sheet 100 moves, and a control part controls the plurality of cutter razors 140 such that the sheet 100 may be cut in a desired form.

Further, press processing, in which cutter razors formed in a desired shape presses while moving up and down, may also be used when necessary.

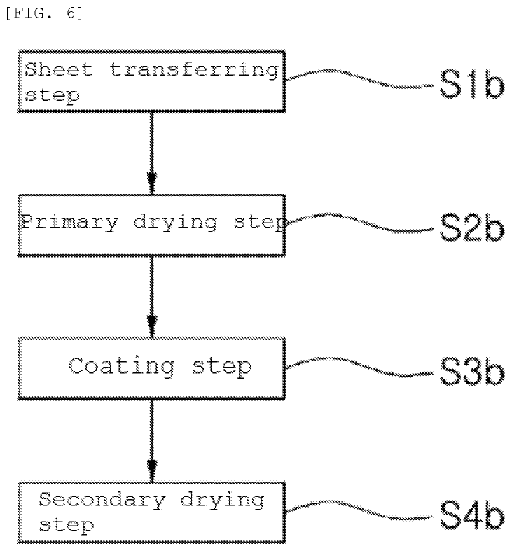

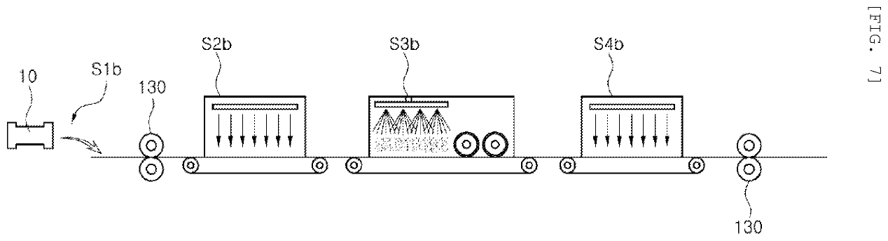

In the above-described processing steps, a nano-coating layer is separately formed and then attached to an absorbent product. Besides these processing steps, a nano-coating layer N may be directly formed on the outer surface of an absorbent product. A method of directly forming a nano-coating layer N on a disposable diaper 10 among absorbent products will be described with reference to FIGS. 6 and 7.

The method includes a diaper transferring step S1b of transferring a disposable diaper 10, to which a pad 11 is not attached, at constant speed and at a constant distance through a conveyor belt 120, a primary drying step S2b of drying an outer surface 12 of the transferred diaper 10, a direct coating step S3b of directly applying a nano-coating material to the one surface of the dried diaper 10, and a secondary drying step S4b of drying the diaper 10 having a nano-coating layer N.

Among the steps in the process illustrated in FIGS. 6 to 7, steps the same as those illustrated in FIGS. 4 to 5 will be omitted. Herein, the direct coating step S3b of directly applying a nano-coating material to the one surface of the dried diaper 10 will be described in detail.

The direct coating step S3b includes preventing damage to an external surface of a diaper and simultaneously preventing movement of the diaper while a nano-coating material is applied because the nano-coating material is directly applied to the outer surface of the diaper. To this end, in the direct coating step S3b, a fixing jig (invisible) is further provided so as to fix the diaper, and in a coating method, roller coating or spray coating etc. is preferably used besides the above-described low-pressure plasma processing. Additionally, roller coating and spray coating may be simultaneously used.

An absorbent product having a nano-coating layer according to the present invention with the above-described configuration may be used for an external patch where pharmaceutical compositions are attached to woven fabric or a disposable bandage etc. in addition to the above-described embodiments.

That is, an absorbent product having a nano-coating layer according to the present invention can help improve air permeability while absorbing and storing exudate etc. coming out of wounds.

### INDUSTRIAL APPLICABILITY

The present invention has been described with respect to the embodiments. It should be understood that the present invention is not limited to what has been described, may be modified and changed by one of ordinary skill in the art to which the present invention pertains in various forms without departing from the technical spirit of the appended claims. Further, such modifications and changes should be construed as being included in the scope of protection of the present invention.

1. An absorbent product having a nano-coating layer, in which an absorbent product, which contacts the body, and absorbs and stores body waste etc. to be excreted or discharged from the body, has a nano-coating layer has a nano-coating layer formed on an external surface of the absorbent product, which is exposed to the outside, so as block absorbed and stored moisture from being discharged to the outside, and to allow circulation of external air, thereby ensuring air permeability.

2. The absorbent product having a nano-coating layer according to claim 1, wherein the nano-coating layer is formed by applying 5 nm to 500 nm, preferably 25 nm to 250 nm of a monomer for nano-coating, which is not harmful to the human body.

3. The absorbent product having a nano-coating layer according to claim 1, wherein the nano-coating layer is formed through any one of knife coating, roller coating, gravure coating, and powder coating, extrusion coating, dip coating, rod coating, spray coating, low-pressure plasma processing.

4. The absorbent product having a nano-coating layer according to claim 3, wherein the low-pressure plasma processing takes place in a vacuum plasma chamber, pressure for performing the processing typically ranges from 10 mTorr to 1000 mTorr, electric power that will be used typically ranges from 5 W to 5000 W, and nano-coating is repetitively performed until the nano-coating layer has a thickness selected from any one of a thickness ranging from 5 nm to 500 nm and preferably, a thickness ranging from 25 nm to 250 nm.

5. An absorbent product having a nano-coating layer and a manufacturing method therefor comprising:
a sheet transferring step of winding a material selected from materials, which do not cause rashes on the body such as pulp, non-woven fabric, usual fiber etc., around a winding roller in the form of a sheet and transferring the sheet at constant speed,
a primary drying step of heating and drying the transferred sheet;
a coating step of applying 5 nm to 500 nm, preferably 25 nm to 250 nm of a monomer for nano-coating, which is not harmful to the human body, to one surface of the dried sheet;
a secondary drying step of drying the sheet whose one surface is nano-coated; and
a cutting step of cutting the sheet such that the sheet has a predetermined length or form.
